(19) United States
(12) Patent Application Publication
(10) Pub. No.: US 2025/0088813 A1
(43) Pub. Date: Mar. 13, 2025

(54) HEARING DEVICE FITTING AGENT WITH CUSTOMIZED ENVIRONMENT MODEL

(71) Applicant: GN Hearing A/S, Ballerup (DK)

(72) Inventors: Tanya IGNATENKO, Eindhoven (NL); Kirill KONDRASHOV, Eindhoven (NL)

(73) Assignee: GN Hearing A/S, Ballerup (DK)

(21) Appl. No.: 18/824,896

(22) Filed: Sep. 4, 2024

(30) Foreign Application Priority Data
Sep. 7, 2023 (EP) 23196116.0
Jan. 15, 2024 (EP) 24151985.9
Apr. 8, 2024 (EP) 24169028.8

(51) Int. Cl. H04R 25/00; A61B 5/12

(52) U.S. Cl. CPC H04R 25/70; A61B 5/123; H04R 25/505; H04R 2225/41

(57) ABSTRACT

A fitting agent for a hearing device system comprising a hearing device, includes one or more processors configured to: initialize a user model comprising a plurality of user preference functions and a user response distribution; obtain environment data indicative of a present environment; determine, using an environment model, an environment state based on the environment data; obtain a test setting comprising primary and secondary test settings; provide the test setting for presentation to the hearing device user; obtain a user input indicative of a preference for either the primary test setting or the secondary test setting as a preferred test setting; update the user model for provision of an updated user model based on the user input and the environment state; and update the environment model for provision of an updated environment model based on the preferred test setting, and based on the user model or the updated user model.

### RELATED APPLICATION DATA

This application claims priority to, and the benefit of, European Patent Application No. 23196116.0 filed on Sep. 7, 2023, European Patent Application No. 24151985.9 filed on Jan. 15, 2024, and European Patent Application No. 24169028.8 filed on Apr. 8, 2024. The entire disclosures of the above applications are expressly incorporated by reference herein.

### FIELD

The present disclosure relates to hearing devices and related tools, methods, and systems in particular for one or more of determining, tuning, fitting and optimizing hearing device parameters and models therefore. Thus, a fitting agent for a hearing device system comprising a hearing device is provided.

### BACKGROUND

Fitting and tuning of hearing devices or hearing aids has always been considered a tedious task of healthcare professionals (HCPs). Traditional approaches for fitting hearing device parameters rely on compensation of a user's hearing loss, based on audiograms, by applying rules such as NAL-NL1 or NAL-NL2. These rules, however, do not take into account specific user preferences.

Recent approaches involve preference learning for hearing devices.

### SUMMARY

Challenges still remain in improving the tools, methods and devices allowing an improved fitting and tuning of hearing device parameters.

A fitting agent is disclosed, the fitting agent optionally being for a hearing device system comprising a hearing device worn by a hearing device user. The fitting agent comprises one or more processors configured to initialize a user model comprising a plurality of user preference functions and associated user response distribution; initialize an environment model; obtain environment data indicative of a present environment; determine, using the environment model, an environment state based on the environment data; obtain a test setting comprising a primary test setting and a secondary test setting for the hearing device based on the environment state; present the test setting to the hearing device user; obtain a user input of a preferred test setting indicative of a preference for either the primary test setting or the secondary test setting; optionally update the user model for provision of an updated user model based on the preferred test setting and the environment state; and update the environment model for provision of an updated environment model based on the preferred test setting and optionally one or more of the user model and the updated user model.

The present disclosure takes into account any differences in user preference in different environments and provides effective and memory-efficient user preference model update. In particular, the need for storing historical and specific user feedback (previous primary and secondary test settings and preference thereof) may be eliminated in turn reducing the memory requirements.

Further, environment-dependent user preference functions may provide simpler user preference functions in turn allowing a simple and effective way to configure one or more hearing device parameters of a hearing device. Further, the present disclosure provides an improved listening experience to the user by improving the modelling of the users preferred hearing parameter settings in different environments in turn resulting in optimized settings being applied in the hearing device, which in turn allows for an improved user experience.

Further, the present disclosure provides an efficient automated search for optimal hearing device parameters by incorporating a user feedback into the learning cycle. A fitting agent is provided, that allows to learn user preferences for hearing device parameters in an efficient and minimally obtrusive way by empowering the user to take direct decisions and have direct impact on the fitting and/or tuning process.

Further, the present disclosure allows that hearing device parameters can be configured, such as fitted and/or tuned, during a normal operating situation and/or with a small number of user inputs/interactions. Thus, a simple and smooth user experience of the hearing device is provided.

It is an advantage of the present disclosure that the hearing device users receive fully personalized settings for their hearing device, based on preferences in the user's own personal customized environments. The user's preferences are used to define and adjust the user's environments and thus also the corresponding hearing device settings or operational modes, which in turn increases user satisfaction and leads to more regular use of the hearing device.

The present disclosure provides for environment personalization/customization and leads to improved system performance. This is achieved by introducing a two-way dependency between environment domain and (user) preference domains providing a significant performance improvement with respect to the baseline model, i.e. a fitting agent without feedback from the user into the environment model.

### BRIEF DESCRIPTION OF THE DRAWINGS

The above and other features and advantages of the present disclosure will become readily apparent to those skilled in the art by the following detailed description of exemplary embodiments thereof with reference to the attached drawings, in which:

FIG. 1 schematically illustrates a hearing system according to the present disclosure,

FIG. 2 illustrates interaction between a user and a fitting agent, and

FIG. 3 is a flow diagram of an exemplary method according to the present disclosure.

### DETAILED DESCRIPTION

Various exemplary embodiments and details are described hereinafter, with reference to the figures when relevant. It should be noted that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

A fitting agent is disclosed. The fitting agent or at least a first part thereof may be implemented, e.g. as an application, in an accessory device, such as an electronic device. The accessory device comprises an interface, a processor, and a memory. The accessory device may for example be or comprise a mobile phone, such as a smartphone, a smart-watch, a special purpose device, a computer, such as a laptop computer or PC, or a tablet computer. The fitting agent or at least a second part thereof may be implemented in a server device. The fitting agent or at least a third part thereof may be implemented in a hearing device. In other words, the fitting agent may be implemented in one or more electronic devices, such as a hearing device and/or accessory device(s).

The present disclosure relates to a hearing device system, fitting agent, accessory device and hearing device of the hearing device system, and related methods. The accessory device forms an accessory device to the hearing device. The accessory device is typically paired or wirelessly coupled to the hearing device. The hearing device may be a hearing aid, e.g. of the behind-the-ear (BTE) type, in-the-ear (ITE) type, in-the-canal (ITC) type, receiver-in-canal (RIC) type, receiver-in-the-ear (RITE) type, or microphone-and-receiver-in-the-ear (MaRIE) type. The hearing device may be a hearable, such as a pair of earbuds or a headset. Typically, the hearing device system is in possession of and controlled by the hearing device user.

The hearing device system may comprise a server device and/or a fitting device. The fitting device is controlled by a dispenser and is configured to determine configuration data, such as fitting parameters. The server device may be controlled by the hearing device manufacturer.

The fitting agent may be a fitting agent for a hearing device system comprising a hearing device worn by a hearing device user. The fitting agent may also be denoted a hearing device fitting agent.

The fitting agent comprises one or more processors. The one or more processors are configured to initialize a user model and an environment model. The environment model is also denoted environmental model. The user model and/or the environment model may be retrieved from a memory of the fitting agent.

The user model comprises a plurality of user preference functions and associated user response distribution. The user response distribution is optionally state-dependent and can be formulated as a single distribution, where depending on the state the distribution has a respective state-dependent form.

The environment model defines or comprises, at least after one step of user feedback, a number of active (observed) environment classes or clusters and/or non-active environment classes or clusters. Each active environment class may correspond to a (single) user preference function. The non-active environment classes are optionally infinite and may be bundled in one single non-active class or cluster. Thereby, spin-out of a new cluster as an active cluster from the non-active cluster is always possible. Thus, the environment model may be said to comprise an infinite number of classes or clusters. Note that class and cluster may be used interchangeably herein.

The user model optionally represents one or a plurality of probabilistic descriptions of user responses, when comparing two sets of hearing device parameter settings. Integral parts of the user model include one or more, such as a plurality of user preference functions and one or more, such as a plurality of distributions of the user responses, also denoted user response distributions, to the presented choices of parameters. The user model comprises one or more distributions of parameters of the respective user preference functions. In other words, each user preference function has an associated distribution of hearing device parameters. In other words, the user model may include a first user preference function and associated first user response distribution, wherein the first user preference function is optionally associated with a first environment class or cluster of the environment model. The user model may include a second user preference function and associated second user response distribution, wherein the second user preference function is optionally associated with a second environment class or cluster of the environment model. The user model may include a third user preference function and associated third user response distribution, wherein the third user preference function is optionally associated with a third environment class or cluster of the environment model.

A vector of hearing device parameters is optionally defined on a D-dimensional continuous compact surface. In particular, hearing device parameters x are optionally defined on a D-dimensional hyper-cube, i.e., x ∈ [0,1]^{D}. In one or more exemplary fitting agents, the hearing device parameters may be normalized by their physical range. The fitting agent, is configured to find optimized/improved values of hearing device parameters, also denoted θ for a particular user. The number D of hearing device parameters may be 1 and/or less than 100, such as in the range from 10 to 50. The number D of hearing device parameters may be larger than 20, such as in the range from 25 to 75.

In general, the user preference functions are unknown. The user preference functions ƒ(x, θ, Λ, k)=ƒ(x; θₖ, Λₖ), k=1, 2, . . . , K, where K is the number of user preference functions, may be parametric functions of hearing device parameters x ∈ [0,1]^{D}, e.g. with known form but unknown shape. This shape is optionally characterized by fitting or tuning parameters, θₖ ∈ [0,1]^{D} and a scaling matrix Λₖ. The scaling matrix Λₖ may be a positive-definite scaling matrix Λₖ. The scaling matrix may be a diagonal matrix Λₖ=diag (λ₁, λ₂, . . . , λ_{M})]), λₘ ∈ [symbol] ⁺, d=1, 2, . . . , D.

The user preference functions or at least one or more of the user preference functions of the K user preference functions may be unimodal.

In one or more exemplary fitting agents, distribution priors may be applied to each element of Λₖ. For example, Lognormal distribution priors may be applied to each element of Λₖ, e.g., log (λ_{d})˜ [symbol] (μ_{d}, σ_{d}²) or Gamma distribution priors may be applied to each element of Λ, e.g., λ_{d}˜Gamma (α_{d}, β_{d}).

The scaling matrix Λₖ does not need to be a diagonal matrix. The scaling matrix Λₖ may be selected as Λₖ=L′*L, where L is a low-triangular matrix (also known as the Cholesky decomposition of Λₖ). Gaussian priors may be applied on each of the elements of L, e.g., Lᵢⱼ˜ [symbol] (μᵢⱼ, σᵢⱼ²).

The maximizing argument θₖ of a k'th environment may be a transformation of another variable, e.g. may be constrained by the following prior assumptions:

$${θ}_{k} = Φ ( \hat{Y} ) , \qquad ( A )$$

where Φ(Ŷ) is a cumulative density function of a probability distribution, such as the standard normal distribution, and Ý is a sample from another probability distribution.

In one or more example fitting agent/methods, the maximizing argument θₖ may be constrained by the following assumptions on representing θₖ as a transformation of Ŷ and corresponding prior distribution of the transformed variable:

$${θ}_{k} = Φ ( \hat{Y} ) , with \hat{Y} ∼ ( {μ}_{k} , {\sum }_{k} ) , \qquad ( B )$$

where Φ(Ŷ)≠_{-∞}^{Ŷ} [symbol] (x|0,1) dx is the cumulative density function of the standard normal distribution, and Ŷ is a sample from the normal distribution with mean vector μₖ and covariance matrix Σₖ. Values of the mean and covariances may be learned from the user responses.

In one or more example fitting agents/methods, the scaling matrix may be a transformed scaling matrix, e.g. by applying a log transform to the elements of the scaling matrix.

The user preference functions may be denoted ƒ or ƒₖ(x; θₖ, Λₖ), where each user preference function ƒₖ(x; θₖ, Λₖ) is associated with environment or state k of K environments/user preference functions. The user preference functions ƒₖ(x; θₖ, Λₖ) may be given by:

$${f}_{k} ( x ; {θ}_{k} , {Λ}_{k} ) = - {( {( x - {θ}_{k} )}^{T} {Λ}_{k} ( x - {θ}_{k} ) )}^{v} , \qquad ( C )$$

where x is a D-dimensional vector optionally in the hypercube [0,1]^{D} and that represents the (D) hearing device parameters of the device in the k'th environment, θₖ is the maximizing argument of ƒₖ, Λₖ is a positive definite D×D scaling matrix characterizing user sensitivity to hearing device parameter changes, D is an integer, and v is a real-valued exponent. The real-valued exponent v may be in the range from 0.01 to 0.99. The real-valued exponent v may be less than 0.5 such as in the range from 0.01 to 0.45. The real-valued exponent v may be larger than 0.5 such as in the range from 0.55 to 0.99. In one or more exemplary fitting agents/methods, the real-valued exponent v may be in the range from 0.25 to 0.75, such as 0.5.

The user preference functions ƒₖ(x; θₖ, Λₖ) may be given by:

$$f ( x ; {θ}_{k} , {Λ}_{k} ) = exp ( - {( x - {θ}_{k} )}^{T} {Λ}_{k} ( x - {θ}_{k} ) ) .$$

The variables θₖ, Λₖ are also denoted user preference function parameters, where θₖ is an optimum of the user preference function that corresponds to the optimal user-specific hearing device parameters and Λₖ ∈ [symbol] ^{D×D} is a diagonal matrix indicative of a user sensitivity, i.e. ability to distinguish change in the parameter. It is noted that other unimodal user preference functions are also possible.

The fitting agent is configured to obtain a primary test setting also denoted x_ref, x^{ref}, or θₜ for the hearing device. The primary test setting x_ref is a vector comprising D hearing device parameters for the hearing device. The hearing device parameters may comprise one or more of filter coefficients, compressor settings, gains, or other parameters relevant for the operation of or signal processing in the hearing device. The primary test setting may be based on and/or dependent on the present environment as indicated by the environment state determined via the environment model. In other words, the primary test setting may be based on and/or dependent on the environment state also denoted c(t), t indicating time step.

The fitting agent is configured to obtain a secondary test setting also denoted x_alt, x^{alt}, or θ′ₜ for the hearing device. The secondary test setting x_ref is a vector comprising D hearing device parameters for the hearing device. The hearing device parameters may comprise one or more of filter coefficients, compressor settings, gains, or other parameters relevant for the operation of or signal processing in the hearing device. The secondary test setting may be based on and/or dependent on the present environment as indicated by the environment state determined via the environment model. In other words, the secondary test setting may be based on and/or dependent on the environment state.

The fitting agent, such as one or more processors of the fitting agent, is configured to present the test setting to the hearing device user. To present the test setting to the hearing device user comprises presenting the primary test setting and/or the secondary test setting to a user, e.g. by transmitting the primary test setting and/or the secondary test setting to the hearing device, the hearing device processing incoming sound in accordance with the primary test setting and/or the secondary test setting for presenting the primary test setting and/or the secondary test setting to the user, and thereby allowing the user to evaluate the test setting. To present the primary test setting and the secondary test setting to a user optionally comprises to output a primary test signal also denoted yt according to the primary test setting. The primary test signal may be an audio signal. The primary test signal may be output via loudspeaker or receiver of a hearing device. To present the primary test setting and the secondary test setting to a user optionally comprises to generate the primary test signal according to the primary test setting in accessory device and to stream the primary test signal from accessory device to hearing device. To present the primary test setting and the secondary test setting to a user optionally comprises to transmit a control signal indicative of primary test signal/primary test setting from accessory device to hearing device. The control signal may include primary test setting. To present the primary test setting and the secondary test setting to a user may comprise to generate the primary test signal according to the control signal in the hearing device, e.g. based on primary test setting of the control signal.

To present the primary test setting and the secondary test setting to a user optionally comprises to output a secondary test signal also denoted y′, according to the secondary test setting. The secondary test signal may be an audio signal. The secondary test signal may be output via loudspeaker or receiver of a hearing device. To present the primary test setting and the secondary test setting to a user optionally comprises to generate the secondary test signal according to the secondary test setting in accessory device and to stream the secondary test signal from accessory device to hearing device. To present the primary test setting and the secondary test setting to a user optionally comprises to transmit a control signal indicative of secondary test signal/secondary test setting from accessory device to hearing device. The control signal may include secondary test setting. To present the primary test setting and the secondary test setting to a user may comprise to generate the secondary test signal according to the control signal in the hearing device, e.g. based on secondary test setting of the control signal.

The fitting agent, such as one or more processors of the fitting agent, is configured to obtain, such as receive and/or detect, a user input of a preferred test setting indicative of a preference for either the primary test setting or the secondary test setting.

The non-preferred test setting is the primary test setting or the secondary test setting not being selected as a preferred test setting. Accordingly, a hearing device and/or accessory device(s) implementing the fitting agent or at least a part of the fitting agent may comprise one or more user interfaces for obtaining, such as receiving and/or detecting, a user input. For example, the hearing device may comprise a user interface receiving a user input. The user interface of the hearing device may comprise one or more buttons, an accelerometer and/or a voice control unit. The accessory device may comprise a user interface. The user interface of the accessory device may comprise a touch sensitive surface, e.g. a touch display, and/or one or more buttons. The user interface of the accessory device may comprise a voice control unit. The user interface of the hearing device may comprise one or more physical sliders, knobs and/or push buttons. The user interface of the accessory device may comprise one or more physical or virtual (on-screen) sliders, knobs and/or push buttons.

The fitting agent is configured to obtain, such as detect, a user input of a preferred/selected test setting indicative of a preference for either the primary test setting or the secondary test setting. In the fitting agent, to detect a user input of a preferred test setting indicative of a preference for either the primary test setting or the secondary test setting may comprise prompting the user for the user input, e.g. by a beep tone signal or voice signal from the hearing device and/or a visual, haptic and/or audio prompt from an accessory device. Detecting a user input may be performed on the hearing device, e.g. by a user activating a button and/or an accelerometer (e.g. single or double tapping the hearing device housing) in the hearing device. To detect a user input may be performed on an accessory device, e.g. by a user selecting a user interface element representative of the preferred test setting, e.g. on a touch-sensitive display of the user accessory device.

In one or more examples, the fitting agent is configured to determine and update hearing device parameters of the hearing device based on the updated user model and optionally the updated environment model.

In one or more examples, a fitting agent for a hearing device system comprising a hearing device worn by a hearing device user is disclosed, wherein the fitting agent comprises one or more processors configured to initialize a user model comprising a plurality of user preference functions and associated user response distribution; initialize an environment model; obtain environment data indicative of a present environment; determine, using the environment model, an environment state based on the environment data; obtain a test setting comprising a primary test setting and a secondary test setting for the hearing device based on the environment state; present the test setting to the hearing device user; obtain a user input of a preferred test setting indicative of a preference for either the primary test setting or the secondary test setting; update the user model for provision of an updated user model based on the preferred test setting and the environment state; and update the environment model for provision of an updated environment model based on the preferred test setting and one of the user model and the updated user model.

In one or more examples, to update the environment model comprises to determine an updated environment state based on one or more of the preferred test setting, the user model, and the updated user model, and to update the environment model based on the updated environment state. In an example, the updated environment state is based on the preferred test setting and the updated user model. Thus, to update the environment model is optionally based on an updated environment state.

In one or more examples, the updated environment state also denoted c′ (t) is indicative of a hard decision or a soft decision. In other words, the updated environment state may be an identifier of an updated environment state (hard decision) or be indicative of an updated environment state distribution (soft decision).

To update the environment model may comprise adjusting a number of active or observed clusters in the environment model.

In one or more examples, to update the environment model comprises to reduce the number of environment classes of the environment model. Reducing the number of environment classes or clusters of the environment model may be done by merging at least two classes of the environment model. For example, two active clusters may be merged to form one active cluster. For example, an active cluster may be discarded by merging the active cluster with the non-active cluster.

In one or more examples, to update the environment model comprises to increase the number of environment classes of the environment model. Increasing the number of environment classes or clusters of the environment model may be done by spin-out or split one or more active classes of the environment model., e.g. by splitting an active environment class into two or more active environment classes or spinning out an active environment class from the non-active environment class.

In one or more examples, the environment model is an infinite Gaussian Mixture Model (GMM).

In one or more examples, to initialize the environment model comprises to obtain, e.g. retrieve from a database, a user profile and initialize the environment model based on the user profile, e,g, by selecting an environment model from a library based on the user profile. The user profile may comprise one or more of age, gender, hearing loss degree, and activity level, and to initialize the environment model is optionally based on one or more of age, gender, hearing loss degree, and activity level.

In one or more examples, the environment model is a hierarchical Dirichlet process Hidden Markov Model (HDP-HMM).

In one or more examples, the environment classes are modelled as latent random variables, e.g. from a countably infinite class space.

In one or more examples, to initialize and/or update the environment model comprises to model environment classes using a Dirichlet process, and wherein each environment class generates a state-specific infinite Dirichlet mixture of Gaussians.

In one or more examples, to update the environment model comprises to apply a Hidden Markov Model, such as a sticky hierarchical Dirichlet process Hidden Markov Model.

The fitting agent/one or more processors of the fitting agent is/are configured to obtain environment data indicative of a present environment and determine, e.g. using the environment model, an environment state based on the environment data.

In one or more example fitting agents, to obtain environment data comprises to obtain audio data and optionally determining the environment data based on the audio data and/or including the audio data in the environment data. In other words, one or more processors of the fitting agent may be configured to obtain audio data and determining the environment data or at least one or more environment parameters based on the audio data. Audio data may comprise first audio data representing or being indicative of audio recorded by one or more microphones of a hearing device of the user. Audio data may comprise second audio data representing or being indicative of audio recorded by one or more microphones of an accessory device or accessory devices of the user. Audio data may comprise third audio data representing or being indicative of audio wirelessly transmitted to a hearing device of the user. For example, the fitting agent may be configured to classify the environment based on hearing device audio and set one or more environment identifiers and/or environment probabilities of the environment data accordingly.

In one or more example fitting agents, to obtain environment data comprises to obtain context data and optionally determining the environment data based on the context data and/or including the context data in the environment data. In other words, one or more processors of the fitting agent may be configured to obtain context data and optionally determining the environment data based on the context data. Context data may be indicative of the context in which the user is in, such as indicative of a user's location, position, movement, temperature, pulse, or other data relevant for the environment. For example, the context data may comprise location data, e.g. GPS coordinates, and/or movement data, such as accelerometer data. The context data may comprise calendar data, and the environment data may be based on the calendar data. The context data may comprise sensor data, e.g. from one or more sensors of an accessory device and/or from one or more sensors of the hearing device. The context data may comprise hearing device data transmitted from the hearing device, such as one or more program identifiers, one or more operating parameters, and/or one or operating mode identifiers of the hearing device.

In one or more example fitting agents, to obtain environment data comprises to receive user input indicative of the environment, e.g. via a user interface of an accessory device. Thus, the user may select and indicate the present environment via accessory device, e.g. from a list of environments presented on a touch-display of the accessory device.

The fitting agent/one or more processors of the fitting agent is/are configured to update the user model, such as one or more user preference functions ƒₖ(x; θₖ, Λₖ) and/or one or more user response distributions Pᵤ, based on hearing device parameters of the preferred test setting and the environment state. In other words, a user response distribution Pᵤ, may model a user response in one or more environments. A user response distribution may be a weighted user response distribution for all or a subset of environments, e.g. where the weights are based on environment probabilities ENVP_k, k=1, . . . , Kₜ (Kₜ is the number of active environment classes at time t) and distributions of parameters of the user preference functions ƒₖ(x; θₖ, Λₖ).

The fitting agent is configured to update the user model based on hearing device parameters of the preferred test setting, a non-preferred test setting and optionally the environment state c(t). In one or more examples, the environment state c(t) is indicative of a hard decision or a soft decision. In other words, the environment state may be an identifier of a state (hard decision) or be indicative of an environment state distribution (soft decision). In particular, the fitting agent may be configured to update the user model based on (r, x_ref, x_alt, c(t)). To update the user model may comprise to update the user preference function, or at least parameters thereof and/or to update parameter distributions associated with user preference functions, based on environment state and/or its reliability (soft) and one or more of the primary test setting, the secondary test setting and the user input of a preferred test setting. In other words, the parameters of the user preference function and/or associated parameter distributions may be updated based on the result of the trial including the primary test setting, the secondary test setting, the preferred test setting of the primary test setting and the secondary test setting, and environment state. To update the user model may comprise to update the user response distribution(s), or at least parameters thereof, based on environment state and one or more of the primary test setting, the secondary test setting and the user input of a preferred test setting. In other words, the parameters of the user preference function may be updated based on the result of the trial including the primary test setting, the secondary test setting, the preferred test setting of the primary test setting and the secondary test setting, and environment data. Thus, to update the user model may comprise to update the parameter distribution(s) of user preference function parameters θₖ and/or Λₖ, based on environment state and one or more of the primary test setting, the secondary test setting and the user input of a preferred test setting.

In one or more exemplary fitting agents and/or methods, to update the user model may be based on Bayesian inference. Updating the user model may comprise updating one or more of the parameters of the user preference function(s) and/or user response distribution(s) and/or parameter distributions associated with the user preference functions. Updating the user model may comprise to determine one or more posteriors of parameters of the user preference function(s).

To update the user model may comprise to determine a posterior of the parameters of one or more of, such as a subset of or all the user preference functions, e.g. based on the environment state, a previous parameter posterior, such as the last or current parameter posterior, preferred test setting, and a non-preferred test setting.

In one or more examples, the fitting agent is implemented as part of a preference learning system as described in the following.

The preference learning system can be modeled as two sub-systems, an agent and a user, interacting with each other in a given context. Its goal is to learn user preferences within the least amount of interactions. The agent represents the trial generating process, given an observed environment. The user represents the internal agent model of the actual user with his/her parameter preferences for different environments.

The user model is optionally characterized by latent unimodal preference functions, parameterized by the environmental state k:

$${f}_{k} ( x ; θ , Λ ) = - \sqrt{{( x - {θ}_{k} )}^{τ} {Λ}_{k} ( x - {θ}_{k} )} , \qquad ( 1 )$$

where x ∈[0,1]^{D} denotes tuning parameters; and θₖ ∈[0,1]^{D} and Λₖ=diag ([λ₁, λ₂, . . . , λ_{M})]), λₘ ∈ [symbol] ⁺, d=1, 2, . . . , D denote the optimal tuning parameters corresponding to the user preference and user sensitivity to parameter changes in state k, respectively1. Here, θ,Λ are collections of all parameters corresponding to all states. Notably, the assumption of x ∈ [0,1]^{D} can be always achieved by normalization.

User interaction with the agent is defined by user evaluation of trials, proposed by the fitting agent. The trial is composed of two parameter options {θref, θalt}, denoted reference proposal (primary test setting) and alternative proposal (secondary test setting), respectively. The user response r to the trial is defined by the binary random variable with r=1, indicating preference for θalt, and r=O, otherwise. The response is based on unknown user preferences.

The user response model that connects binary user responses to continuous valued tuning parameters and takes into account user uncertainty about his/her preferences is known as binomial-probit regression model. We extend this model to a state-dependent user response model:

$$P ( r \vert {θ}^{ref} , {θ}^{alt} , θ , Λ , c = k ) = Ber ( r \vert Φ ( {f}_{k} ( {θ}^{alt} ; θ , Λ \} - {f}_{k} ( {θ}^{ref} ; θ , Λ ) ) ) , \qquad ( 2 )$$

where c ∈ N is an environmental state, {θref, θalt} is the trial for this state, and Ber (·|·) and Φ(·) denote Bernoulli probability mass function (PMF) and standard normal cumulative distribution function (CDF), respectively.

Parameters θ and Λ (collections of all θₖ and Λₖ parameters) of the user preference function (1) are unknown latent random variables that have to be learned through interactions between the user and the agent. To operate in the unconstrained parameter domain, parameters are transformed and redefined as ϕ=[Φ⁻¹(θ), ln Λ]^{T}, where ϕ is a collection of ϕₖ for all states k, and transformation functions are applied to the parameters element-wise. Parameter priors are assumed to have Gaussian distribution: p(ϕₖ)= [symbol] (ϕₖ|μ_{ϕ}, Σ_{ϕ}).

To infer these parameters, the agent may collect the most informative observations by generating the trials, responses to which lead to the highest model uncertainty reduction.

Then the context-dependent preference learning system is defined by two components: one corresponding to the generative trial distributions Pₜ^{α}, given previous observations and trials; and the other to the predictive user response distributions Pₜ^{u}, used by the agent to model the user response, i.e.,

$${P}_{t}^{a} = p ( \{ {θ}_{t ,}^{ref} , {θ}_{t}^{alt} \} \vert ϕ , {c}_{1 : t} , {r}_{1 : t - 1} , {\{ {θ}^{ref} , {θ}^{alt} \}}_{1 : t - 1} ) \\ \begin{matrix}{P}_{t}^{u} = P ( {r}_{t} \vert ϕ , {c}_{1 : t} , {r}_{1 : t - 1} , {\{ {θ}^{ref} , {θ}^{alt} \}}_{1 : t - 1} , \{ {θ}_{t}^{ref} , {θ}_{t}^{alt} \} ) \\ = P ( {r}_{t} \vert ϕ , {c}_{t} , \{ {θ}_{t}^{ref} , {θ}_{t}^{alt} \} ) .\end{matrix} \qquad ( 3 )$$

It is noted that the fitting agent/preference learning system depends on the environment state cₜ. It is assumed that each environmental state k corresponds to a single preference function. The environment state cₜ, such as environment identifier or environment state distribution, is determined via the environment model and fed to the user model/preference learning system, e.g. for trial design.

To model the environment, infinite Bayesian Gaussian mixture model (GMM) approach is taken, then

$$p ( {s}_{t} \vert m , W , {c}_{t} = k ) = ( {s}_{t} \vert {m}_{k} , {W}_{k}^{- 1} ) , \qquad ( 4 )$$

where mₖ and Wₖ are mean vector and precision matrix of kth mixture component, respectively, and their priors are assumed to have a Normal-Wishart distribution with hyperparameters m₀, λ₀, W₀, v₀:

$$p ( {m}_{k} , {W}_{k} ) = ( {m}_{k} , {W}_{k} \vert {m}_{0} , {λ}_{0} , {W}_{0} , {ν}_{0} ) . \qquad ( 5 )$$

Further, for a mixing probability vector It, the component or cluster label cₜ follows categorical distribution

$$p ( {c}_{t} \vert π ) = Cat ( {c}_{t} \vert π ) . \qquad ( 6 )$$

Lastly, the prior for It must be defined. Note that so far, a number of mixture components in the GMM have not been defined. Indeed, this number is unknown a priori and changes over time during evolution of the preference leaning system in user-agent interactions. Mixing components may be generated by DP, i.e., it is assumed K=∞ components with mixing prior given by GEM distribution:

$$p ( π ) = GEM ( π \vert α ) = {lim}_{K \to ∞} Dir ( α / K , … ) . \qquad ( 7 )$$

Summing up, combining the environment model with the state-depended preference learning system (user model), a context-aware preference learning system defined by the following generative model is obtained:

$$π ∼ GEM ( α ) \qquad ( 8 a )$$

$${c}_{t} \vert π ∼ Cat ( π ) \qquad ( 8 b )$$

$${m}_{k} , {W}_{k} ∼ ( {m}_{0} , {λ}_{0} , {W}_{0} , {ν}_{0} ) \qquad ( 8 c )$$

$${s}_{t} \vert {c}_{t} = k , m , W ∼ ( {m}_{k} , {W}_{k}^{- 1} ) \qquad ( 8 d )$$

$${ϕ}_{k} ∼ ( {μ}_{ϕ} , {\sum }_{ϕ} ) \qquad ( 8 e )$$

$$\{ {θ}_{t}^{ref} , {θ}_{t}^{alt} \} \vert {c}_{t} = k , ϕ ∼ {P}_{t}^{a} \qquad ( 8 f )$$

$${r}_{t} \vert \{ {θ}_{t}^{ref} , {θ}_{t}^{alt} \} , {c}_{t} = k , ϕ ∼ Ber ( Φ ( {f}_{k} ( {θ}_{t}^{alt} ; θ , Λ ) - {f}_{k} ( {θ}_{t}^{ref} ; θ , Λ ) ) ) . \qquad ( 8 g )$$

In an example implementation, parameters are sequentially infered and passed through the system starting from the environment model to the user preference model; and, after the trial, re-evaluate the current model state and update the models, but now in the opposite direction: from user preference domain (user model) to the environmental domain (environment model). The sequential updating and greedy search (SUGS) algorithm may be adopted for DP-mixtures for the environment clustering step. The cluster labels or active environment classes are optionally selected greedily by maximizing the cluster posterior.

The model specified by (8a)-(8d) is used for estimating the initial cluster label or environment state cₜ⁰ as

$${c}_{t}^{0} = {arg max}_{k} {{q}_{t} ( {c}_{t} = k )}_{t} , \qquad ( 9 )$$

where qₜ(cₜ) is the approximate posterior given by

$${q}_{t} ( {c}_{t} ) ∝ p ( {s}_{t} \vert {c}_{t} ) {p}_{t} ( {c}_{t} ) , \qquad ( 10 )$$

with pₜ(cₜ) representing the prior of the relative occurrence of each environment:

$${p}_{t} ( {c}_{t} = k ) = \{ \begin{matrix}\frac{{n}_{t - 1}^{k}}{t - 1 + α} & if k \leq {K}_{t - 1} \\ \frac{α}{t - 1 + α} & if k = {K}_{t - 1} + 1\end{matrix} , \qquad ( 11 )$$

where nₜ₋₁^{k} is the number of data points, assigned to the cluster or environment state k and Kₜ₋₁ is the total number of explored environment states until time t−1. The evidence term can be computed as

$$p ( {s}_{t} \vert {c}_{t} = k ) = \frac{1}{{π}^{b / 2}} \frac{{Γ}_{b} ( {ν}_{k , t} / 2 )}{{Γ}_{b} ( {ν}_{k , t - 1} / 2 )} \frac{{\vert {W}_{k , t} \vert}^{{ν}_{k , t} / 2}}{{\vert {W}_{k , t - 1} \vert}^{{ν}_{k , t - 1} / 2}} {( \frac{{λ}_{k , t - 1}}{{λ}_{k , t}} )}^{b / 2} , \qquad ( 12 )$$

where Bayesian rule was applied, given the chosen conjugate prior (5). Here b is the dimensionality of sₜ.

Next, the user preference model priors are specified by (8e)-(8g). Optionally, assumed density filtering can be used to obtain an approximate preference posterior:

$${p}_{t} ( ϕ \vert {r}_{1 : t} , {c}_{t}^{0} = k , {c}_{1 : t - 1}^{*} , {\{ {θ}^{ref} , {θ}^{alt} \}}_{1 : t} ) = ( ϕ \vert {μ}_{ϕ , k} , {\sum }_{ϕ , k} ) \cdot {\prod }_{{t}^{′} ∈ \{ \{ 1 , … , t - 1 \} : {c}_{{t}^{′}}^{*} = k , t \}} \frac{Ber ( {r}_{{t}^{′}} \vert Φ ( {f}_{k} ( {θ}_{{t}^{′}}^{alt} ; ϕ ) - {f}_{k} ( {θ}_{{t}^{′}}^{ref} ; ϕ ) ) )}{{{t}^{′} , k}_{}} \approx ( {ϕ}_{k} \vert {μ}_{ϕ , t}^{k} , {\sum }_{ϕ , t}^{k} ) = {q}_{t} ( {ϕ}_{k} ) , \qquad ( 13 )$$

for cluster cₜ⁰=k at time t, were c_{1:t-1}* denotes cluster assignments up to time t. To apply this, the relevant data must be collected. Therefore, an efficient trial design strategy may be specified. First, we present a straightforward agent strategy that can be used for each individual environment and then propose its variant that incorporates cluster reliability information. The trial design begins with the initial trial (θ₀^{ref}), θ₀^{alt}), e.g. selected at random. The agent strategy for the efficient sequential trial design, aiming at fast reduction of model uncertainty, is defined as

$$\{ {θ}_{t , k}^{ref} , {θ}_{t , k}^{alt} \} = {arg max}_{\{ {θ}_{t , k}^{ref} , {θ}_{t , k}^{alt} \} ∼ {P}_{t , k}^{a}} I ( {R}_{t} ; {ϕ}_{k} \vert {1 : t - 1}_{} , \{ {θ}_{t , k}^{ref} \cdot {θ}_{t , k}^{alt} \} , {c}_{t} = k , {c}_{1 : t - 1}^{*} ) , {P}_{t , k}^{a} = {δ ( {θ}_{t}^{ref} - {θ}_{t - 1}^{alt} )}^{{r}_{t - 1}} {δ ( {θ}_{t}^{ref} - {θ}_{t - 1}^{ref} )}^{1 - {r}_{t - 1}} {q}_{t - 1}^{k} ( {θ}_{t}^{alt} ) , \qquad ( 14 )$$

where I (·;·) denotes mutual information, Rₜ response RV, [symbol] {θₜ^{ref}, θₜ^{alt}, rₜ} is trial results at time t, and qₜ₋₁^{k}(·) is the marginalized posterior (13) over sensitivity parameter.

The above trial design is based on the hard decision about the cluster assignment. In an example, it is proposed to incorporate soft information about cluster assignment into the design of an alternative proposal. The alternative proposal is then given by

$${θ}_{t}^{alt} = {\sum }_{k = 1}^{{K}_{t} + 1} q ( {c}_{t}^{0} = k ) \cdot {θ}_{t , k}^{alt} , \qquad ( 15 )$$

where θ_{t,k}^{alt} are generated by k=1, 2, . . . , Kₜ agents according to (14), while θ_{t,K}_{ₜ₊₁}^{alt} by a new unexplored agent and thus is sampled from the preference priorAfter the trial, given the new data, we need to update our models.

It is to noted that an alternative trial design may be applied by a maximization of weighted mutual information based on probability distribution of environment clusters.

The user response allows for a re-evaluation of the cluster assignment decision (estimation of environment state), by evaluating posterior membership of the new reference θ_{t+,k}^{ref} also called personalized environmental state. Then the updated personalized environmental state is given by

$${c}_{t}^{1} = {arg max}_{k} {q}_{t} ( {c}_{t}^{1} = k ) , \qquad ( 16 )$$

where approximate posterior distribution of cₜ¹ is given by the preference mixture

$${q}_{t} ( {c}_{t}^{1} ) ∝ p ( {θ}_{t + 1 , k}^{ref} \vert {c}_{t}^{1} ) {p}_{t} ( {c}_{t}^{1} ) , \qquad ( 17 )$$

here p(θ_{t+1,k}^{ref}|Cₜ¹=k)=qₜ(θ_{t+1,k}^{ref} with qₜ(·) being, as before, marginalized parameter posterior, and pₜ(cₜ¹) being the prior of the relative occurrence of each personalized cluster; or the prior that is the posterior of the environmental state from the environmental model. Finally, the new environment state cₜ¹ is used as the cluster label of the environment data, e.g signal feature sₜ and for update of both the environment model using sₜ and the preference distribution (13) for cₜ¹ using Dₜ. Alternatively, to incorporate soft (reliability) information, qₜ(·) can be used to update the user model and/or the environment model.

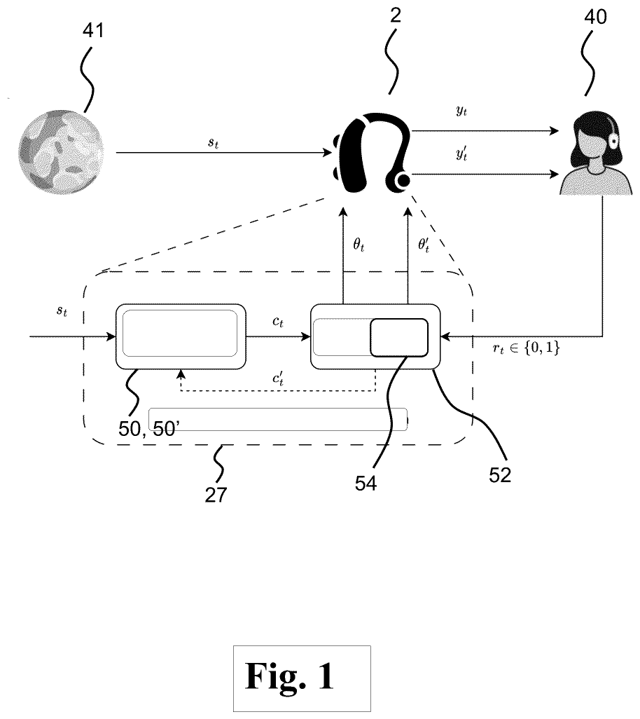

FIG. 1 shows a schematic overview of the fitting agent with a context-aware preference learning system. A signal sₜ is received by the hearing device 2 and in the fitting agent 27 as environment data, e.g. implemented in accessory device, the fitting agent passing the environment data to the environment model 50. The environment model 50 determines the environment state cₜ, such as environment identifier and/or environment distribution associated with environment 41, based on environment data including audio input signal sₜ, and passes the environment state to the preference learning system 52. Based on the environment state and previous trials and user responses, the preference learning system 52 including user model 54 proposes two tuning parameter sets θ (primary test setting) and e′ (secondary test setting) for the hearing device. The hearing device 2 processes the signal sₜ observed by microphone(s) of the hearing device 2 using these tuning parameters θ and θ′ to generate audio signals y and y′, respectively, which are then presented to the user 40 via receiver of the hearing device for evaluation of the test setting. The user 40 indicates preference for the primary test setting or secondary test setting via user input represented by r that is fed back to the preference learning system 52. The preference learning system 52 updates the user model 54 and feeds an updated environment state cₜ′, such as updated environment identifier and/or updated environment distribution associated with environment 41, back to the environment model for update of the environment model for provision of an updated environment model 50′ based on the preferred test setting and one of the user model or the updated user model.

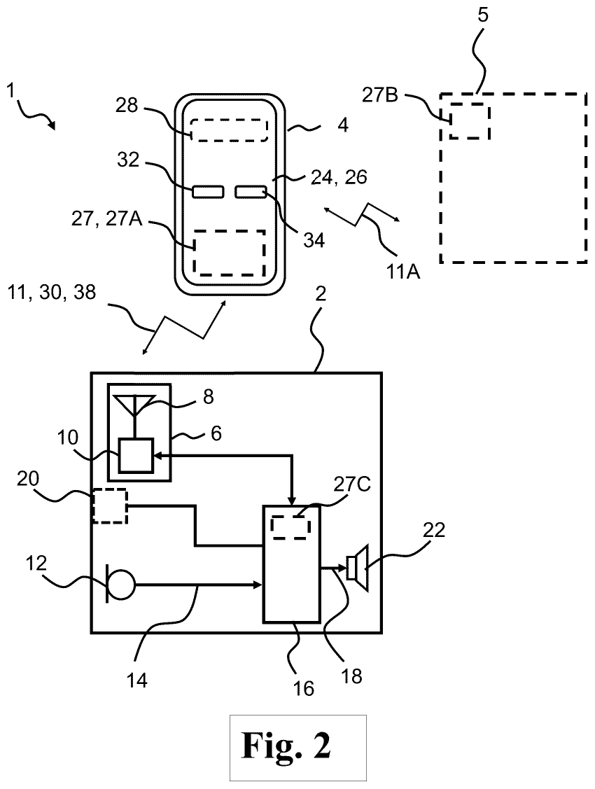

FIG. 2 is an overview of a hearing system with a fitting agent according to the present disclosure. The hearing system comprises a hearing device 2, an accessory device 4, and optionally a server device/fitting device 5. The hearing device 2 comprises a transceiver module 6 for (wireless) communication with the accessory device 4 and optionally a contralateral hearing device (not shown in FIG. 1). The transceiver module 6 comprises antenna 8 and transceiver 10 and is configured for receipt and/or transmission of wireless signals via wireless connection 11 to the accessory device 4. The accessory device 4 is configured for receipt and/or transmission of wireless signals via wireless connection 11A to the server device/fitting device 11A. The hearing device 2 comprises a set of one or a plurality of microphones comprising a first microphone 12 for provision of a first microphone input signal 14; a processor 16 for processing input signals including the first microphone input signal 14 according to one or more hearing device parameters and providing an electrical output signal 18 based on input signals; an optional user interface 20 connected to the processor 16; and a receiver 22 for converting the electrical output signal 18 to an audio output signal.

The accessory device 4 is a smartphone and comprises a user interface 24 comprising a touch display 26, a processor (not shown), and a memory (not shown).

In the hearing device system 1, the fitting agent 27 is an application installed in the memory of the accessory device 4.

The fitting agent 27 is a fitting agent for update of a user model and/or an environment model for a hearing device user and/or one or more of optimizing, determining, fitting, tuning, and modelling hearing device parameters of a hearing device.

The fitting agent 27 comprises one or more processors, such as one or more processors of accessory device and hearing device, configured to initialize a user model and an environment model, the user model comprising a plurality of user preference functions and associated user response distributions, wherein each user preference function ƒ(x; θₖ, Λₖ) is associated with a k'th environment (k=1, 2, . . . , Kₜ); initialize an environment model defining a number of environment classes; obtain environment data indicative of a present environment; determine, using the environment model, an environment state based on the environment data; obtain a test setting comprising a primary test setting and a secondary test setting for the hearing device based on the environment state; present the test setting to the hearing device user; obtain a user input of a preferred test setting indicative of a preference for either the primary test setting or the secondary test setting; update the user model for provision of an updated user model based on the preferred test setting and the environment state; and update the environment model for provision of an updated environment model based on the preferred test setting and the updated user model.

The fitting agent 27 implemented in the accessory device 4 is configured to update the user model and/or the environment model, such as a plurality of user preference functions of the user model. In other words, the fitting agent is configured to update the user model based on hearing device parameters of the preferred test setting and the environment data. To update the user model may comprise updating the user preference models and the user response models based on the primary test setting, the secondary test setting, the preferred test setting, and the environment state. The fitting agent 27/accessory device 4 may be configured to transmit the primary test setting, the secondary test setting, and the preferred test setting to server device 5 that updates the user model and/or the environment model and transmits the updated user model and/or environment model to the fitting agent 27/accessory device 4. Thus, fitting agent 27/accessory device 4 may be configured to receive the updated models from the server device 5. In other words, the fitting agent 27 may be distributed on accessory device 4 and one or more of hearing device 2 and server device 5. Thus, the fitting agent 27 may comprise a first part 27A implemented in accessory device 4, optional second part 27B implemented in server device, and optional third part 27C implemented in hearing device 2, such as in processor 16.

In one or more example fitting agents including fitting agent 27, 27A, 27B, 27C, to obtain environment data comprises to obtain audio data and determining the environment data based on the audio data. For example, microphone 12 of the hearing device 2 may provide audio data representative of the sound received by the microphone 12, wherein the audio data can be transmitted to the accessory device 4 wherein the fitting agent 27, 27A of the accessory device 4 is configured for determining the environment data based on the audio data from the hearing device and/or wherein the fitting agent 27C of the hearing device 2 is configured for determining the environment data or at least a part of the environment data based on the audio data from microphone 12. The environment data determined by fitting agent 27C of the hearing device 2 is transmitted to the fitting agent 27A of the accessory device 4 for further processing.

In one or more example fitting agents including fitting agent 27, 27A, 27B, 27C, to obtain environment data comprises to obtain context data, e.g. from one or more sensors of the hearing device 2 and/or accessory device 4, and determining the environment data based on the context data. Determining the environment data may be based on the audio data and the context data. In one or more example fitting agents including fitting agent 27, 27A, 27B, 27C, to obtain context data comprises to obtain context data from one or more applications, such as a calendar application, of the accessory device 4.

In one or more example fitting agents including fitting agent 27, 27A, 27B, 27C, to obtain a test setting comprising a primary test setting and a secondary test setting for the hearing device comprises to determine the secondary test setting based on the environment state.

In one or more example fitting agents including fitting agent 27, 27A, 27B, 27C, the fitting agent is configured to determine and update hearing device parameters of the hearing device based on the updated user model and optionally the updated environment model.

In an implementation including accessory device 4, the fitting agent 27/accessory device 4 may be configured to send a control signal 30 to the hearing device 2, the control signal 30 being indicative of the primary test setting and the secondary test setting, thus enabling the hearing device 2 to output test signals accordingly.

The hearing device 2 (processor 16) is optionally configured to output a primary test signal according to the primary test setting via the receiver 22 and a secondary test signal according to the secondary test setting via the receiver 22.

The fitting agent 27, 27A, 27C (hearing device 2 (processor 16) and/or the accessory device 4) is configured to detect a user input of a preferred test setting indicative of a preference for either the primary test setting or the secondary test setting, e.g. by detecting a user input on user interface 20 or by detecting a user selection of one of a primary virtual button 32 (primary test setting is preferred) and a secondary virtual button 34 (secondary test setting is preferred) on the user interface 24 of accessory device 4.

It is to be noted that the fitting agent 27, 27A, 27B, 27C may be configured to detect a user input of a preferred test setting indicative of a preference for either the primary test setting or the secondary test setting, e.g. by receiving a wireless input signal from a secondary accessory device, such as smartwatch comprising a user interface. Thereby, a more convenient user input is provided for, which in turn increases the user friendliness of the fitting agent.

A user may initiate update of the user model and/or the environment model by pressing start button 28 on the user interface of accessory device 24. In other words, the fitting agent may detect user input indicative of start and perform the method/update of the user model in accordance with detection of the user input indicative of start.

In an implementation including accessory device 4, the accessory device 4 may be configured to send a control signal 38 to the hearing device 2, the control signal 38 being indicative of the hearing device parameters of the preferred test setting, thus enabling the hearing device to update and apply preferred hearing device parameters in the hearing device.

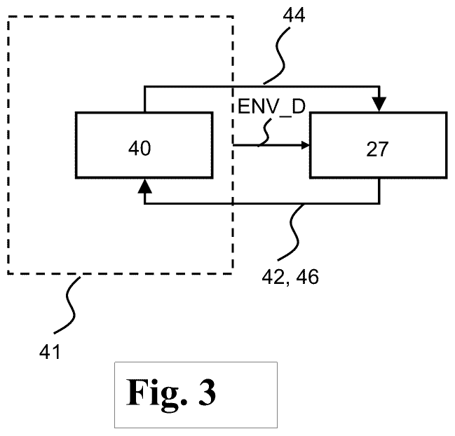

FIG. 3 illustrates interaction between a user 40 in an environment 41 and a fitting agent 27. The fitting agent 27 obtains environment data ENV_D indicative of the present environment 41, e.g. based on audio data and/or context data from one or more hearing devices worn by user 40, from user providing user input to fitting agent, from accessory device microphone, sensors, and/or applications.

For each interaction or trial indexed with index n, the fitting agent generates a primary test setting xₙ^{ref} and a secondary test setting xₙ^{alt} and presents 42 the test settings xₙ^{ref} and xₙ^{alt} for the user, e.g. by controlling hearing device for outputting primary test signal and secondary test signal respectively according to and indicative of the primary test setting and the secondary test setting. The user evaluates the two test settings xₙ^{ref} and xₙ^{alt}, and the fitting agent 27 receives and detects the user's response 44, In indicative of the preferred test setting of the primary test setting and the secondary test setting. The fitting agent updates the user model based on environment state Cₜ, rₙ, xₙ^{ref}, and xₙ^{alt} salt and subsequently updates the environment model based on the updated user model.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Memory may be one or more of a buffer, a flash memory, a hard drive, a removable media, a volatile memory, a non-volatile memory, a random access memory (RAM), or other suitable device. In a typical arrangement, memory may include a non-volatile memory for long term data storage and a volatile memory that functions as system memory for the processor. Memory may exchange data with processor over a data bus. Memory may be considered a non-transitory computer readable medium.

Memory may be configured to store information in a part of the memory.

Furthermore, the labelling of a first element does not imply the presence of a second element and vice versa.

It may be appreciated that the figures comprise some modules or operations which are illustrated with a solid line and some modules or operations which are illustrated with a dashed line. The modules or operations which are comprised in a solid line are modules or operations which are comprised in the broadest example embodiment. The modules or operations which are comprised in a dashed line are example embodiments which may be comprised in, or a part of, or are further modules or operations which may be taken in addition to the modules or operations of the solid line example embodiments. It should be appreciated that these operations need not be performed in order presented. Furthermore, it should be appreciated that not all of the operations need to be performed. The exemplary operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It is to be noted that the word “based on” may be seen as “as a function of” and/or “derived from”. The terms “based on” and “as a function of” can be used interchangeably. For example, a parameter determined “based on” a data set can be seen as a parameter determined “as a function of” the data set. In other words, the parameter may be an output of one or more functions with the data set as an input.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, and systems described herein are described in the general context of method steps processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

1 hearing device system
2 hearing device
4 accessory device
5 server device
6 transceiver module
8 antenna
10 transceiver
11 wireless connection between hearing device and accessory device
11A wireless connection between accessory device and server device
12 first microphone
14 first microphone input signal
16 processor
18 electrical output signal
20 user interface
22 receiver
24 user interface of accessory device
26 touch display
27 fitting agent
27A first part of fitting agent
27B second part of fitting agent
27C third part of fitting agent
28 (virtual) start button
30 control signal indicative of primary and secondary test setting
32 primary virtual button
34 secondary virtual button
38 control signal indicative of the hearing device parameters of the preferred test setting
40 user
41 environment
42 n'th trial, test settings xₙ^{ref} and xₙ^{alt}
44 user response to n'th trial
46 n+1'th trial, test settings xₙ₊₁^{ref} and xₙ₊₁^{alt}
50 environment model
52 preference learning system
54 user model

1. A fitting agent for a hearing device system comprising a hearing device to be worn by a hearing device user, wherein the fitting agent comprises one or more processors configured to:
initialize a user model comprising a plurality of user preference functions and a user response distribution;
obtain environment data indicative of a present environment;
determine, using an environment model, an environment state based on the environment data;
obtain a test setting comprising a primary test setting and a secondary test setting for the hearing device based on the environment state;
provide the test setting for presentation to the hearing device user;
obtain a user input indicative of a preference for either the primary test setting or the secondary test setting as a preferred test setting;
update the user model for provision of an updated user model based on the user input and the environment state; and
update the environment model for provision of an updated environment model based on the preferred test setting, and based on the user model or the updated user model.

2. The fitting agent according to claim 1, wherein the fitting agent is configured to determine an updated environment state based on the user model or the updated user model, and to update the environment model based on the updated environment state.

3. The fitting agent according to claim 1, wherein the fitting agent is configured to update the environment model by reducing a number of environment classes of the environment model.

4. The fitting agent according to claim 1, wherein the fitting agent is configured to update the environment model by increasing a number of environment classes of the environment model.

5. The fitting agent according to claim 1, wherein the environment model is an infinite Gaussian Mixture Model.

6. The fitting agent according to claim 1, wherein the fitting agent is configured to obtain a user profile, and to initialize the environment model based on the user profile.

7. The fitting agent according to claim 6, wherein the user profile comprises one or more of age, gender, hearing loss degree, and activity level, and wherein the fitting agent is configured to initialize the environment model based on the one or more of age, gender, hearing loss degree, and activity level.

8. The fitting agent according to claim 1, wherein the environment model is a hierarchical Dirichlet process Hidden Markov Model (HDP-HMM).

9. The fitting agent according to claim 1, wherein environment classes of the environment model are modelled as latent random variables from a countably infinite class space.

10. The fitting agent according to claim 1, wherein the fitting agent is configured to use a Dirichlet process to model environment classes of the environment model.

11. The fitting agent according to claim 1, wherein the environment model comprises environment classes, and wherein each of the environment classes is associated with a state-specific infinite Dirichlet mixture of Gaussians.

12. The fitting agent according to claim 1, wherein the fitting agent is configured to update the environment model by applying a sticky hierarchical Dirichlet process Hidden Markov Model.

13. The fitting agent according to claim 1, wherein the environment state is an environment identifier indicative of the present environment.

14. The fitting agent according to claim 1, wherein the environment state is a probability distribution for the present environment.

15. The fitting agent according to claim 1, wherein the fitting agent is also configured to initialize the environment model.
